(19) United States
(12) Patent Application Publication
(10) Pub. No.: US 2018/0203009 A1
(43) Pub. Date: Jul. 19, 2018

(54) DEVICE, SYSTEM AND METHOD FOR MANAGING TREATMENT OF AN INFLAMMATORY AUTOIMMUNE DISEASE OF A PERSON

(71) Applicant: KONINKLIJKE PHILIPS N.V., EINDHOVEN (NL)

(72) Inventors: Vanda Lucia de Carvalho VITTORINO DE ALMEIDA, Veldhoven (NL); Sima ASVADI, Eindhoven (NL)

(21) Appl. No.: 15/743,877

(22) PCT Filed: Jul. 14, 2016
(86) PCT No.: PCT/EP2016/066708

§ 371 Date: Jan. 11, 2018

(30) Foreign Application Priority Data
Jul. 16, 2015 (EP) 15177012.0

(51) Int. Cl. G01N 33/564; G06F 19/18; A61B 5/145

(52) U.S. Cl. CPC G01N 33/564; G06F 19/18; A61B 5/0008; G01N 2800/102; G01N 2800/52; A61B 5/14546

(57) ABSTRACT

The present invention relates to a device, system and method for managing treatment of an inflammatory autoimmune disease of a person. The proposed device comprises an input unit (20) for receiving measurement data acquired over time including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration. The device further comprises a treatment manager (21) for managing treatment of the inflammatory autoimmune disease of the person by combining the obtained measurement data by an algorithm to obtain a disease activity level, to monitor the disease activity level over time and to determine treatment interventions based on the monitored disease activity level.

### FIELD OF THE INVENTION

The present invention relates to a device, system and method for managing treatment of an inflammatory autoimmune disease, such as Rheumatoid Arthritis, of a person.

### BACKGROUND OF THE INVENTION

Biological processes and functions at all hierarchical levels are organized in time as biological rhythms of discrete periods. Circadian (24-hour) rhythms, which are of direct importance to clinical medicine, are orchestrated by a set of clock genes of the master brain clock situated in the suprachiasmatic nuclei of the hypothalamus plus numerous subservient peripheral cellular clocks of all tissues and organs.

As is known from Haus, Erhard et al., Rheumatoid Arthritis Circadian Rhythms in Disease Activity, Signs and Symptoms, and Rationale for Chronotherapy with Corticosteroids and Other Medications, Bulletin of the NYU Hospital for Joint Diseases 2012; 70 (Suppl 1):3-10, the organization and communications of human biologic processes and functions entail a complex web, the components being the central nervous system with its sympathetic and parasympathetic branches, glandular endocrine system, peripheral endocrine tissues (e.g., adipose tissue and intestinal tract), and immune system. All components of this web are discretely organized in time in the form of a multi-frequency time structure, with optimal functioning (i.e. “health”) being dependent on the well-adapted interactions of rhythmic variables.

The circadian time structure gives rise to in-time predictable patterns for day and night in morbid and mortal events plus symptom occurrence and severity of common chronic conditions, including rheumatoid arthritis (RA). The 24-hour variation in RA symptoms, such as joint pain, morning stiffness, and functional disability, in large part is due to predictable-in-time differences during the 24 hours in the circulating concentration of the anti-inflammatory hormone cortisol relative to the circadian rhythm of key disease-exacerbating and pro-inflammatory cytokines. Plasma cortisol peaks during the daytime activity span; however, during the late night and early morning, plasma cortisol level is markedly reduced and thus unable to counter the increased RA disease activity signaled nocturnally by interleukin-6 or (IL-6), tumor necrosis factor-alpha (TNF-α), and various other inflammatory cytokines.

The cortisol circadian rhythm of RA patients with low or moderate activity remains normal. However, it can be disturbed—decrease of rhythm amplitude and elevated cortisol concentrations, often with double peaks in the morning and during the afternoon and evening daily “quiet period” of the adrenal—in RA patients with high disease activity. Cortisol concentrations in RA patients with high or advanced disease activity tend to be elevated but without sufficient effect to counter the pathological remodeling of affected tissues.

Melatonin and prolactin, produced primarily during the night time sleep span, up-regulate the immune system, resulting in increase of the T helper cell 1 (Th1) inflammatory response and RA disease activation when cortisol values are lowest. In this regard, higher nighttime serum concentrations of melatonin and prolactin have been reported in patients with active RA.

Circadian system-dependent immune processes interact with sleep-dependent biological functions, which are significantly altered by sleep deprivation. Pro-inflammatory cytokines—IL-6, IL-1, and TNF-α, which are partially controlled by sleep—themselves exert regulatory influences on both the immune system and sleep mechanisms. IL-6, a biomarker of RA disease activity, is circadian rhythmic; it attains peak plasma levels in the late night and early morning hours.

The actions of the pro-inflammatory cytokine TNF-α are further amplified by the sleep-dependent, high-amplitude variation of soluble TNF receptors I and II, thereby extending the trans-signaling effect of pro-inflammatory cytokines to a broader group of cells also outside the immune system. Production of pro-inflammatory cytokines is maximal during nocturnal sleep, whereas production of anti-inflammatory cytokines is maximal during diurnal wakefulness. In RA patients, the peak time of both cytokines is shifted to the morning, with markedly elevated peak concentration of TNF-α found in most studies at approximately 6 a.m. and that of IL-6 found at approximately 7 a.m., the result being markedly elevated amplitude of their circadian patterning with extension of their elevated levels later during the daytime wake span.

Thomas Bollinger et al.: “The influence of regulatory T cells and diurnal hormone rhythms on T helper cell activity”, IMMUNOLOGY, vol. 131, no. 4, 15 Jul. 2010 (2010-07-15), pages 488-500 demonstrated that interleukin (IL)-2, interferon-γ (IFN-γ), tumour necrosis factor-α (TNF-α) and IL-10 secretion by naïve CD4⁺ T cells follows a diurnal rhythm. Furthermore, multiple regression analysis, as well as subsequent in vitro experiments, suggested that serum levels of cortisol and prolactin are part of the underlying mechanism.

### SUMMARY OF THE INVENTION

It is an object of the present invention to provide an improved, personalized device, system and method for managing treatment of an inflammatory autoimmune disease, such as Rheumatoid Arthritis, of a person.

In a first aspect of the present invention a device for managing treatment of an inflammatory autoimmune disease of a person is presented, said device comprising
an input unit for receiving measurement data acquired over time including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration, and
a treatment manager for managing treatment of the inflammatory autoimmune disease of the person by combining the obtained measurement data by an algorithm to obtain a disease activity level, to monitor the disease activity level over time and to determine treatment interventions based on the monitored disease activity level.

In a further aspect of the present invention a corresponding method is presented.

In yet a further aspect of the present invention a system for managing treatment of an inflammatory autoimmune disease of a person is presented, said system comprising:
a white blood cell counter for counting the white blood cells of the person,
a tumor necrotic factor-α sensor for determining the tumor necrotic factor-α data related to the person's tumor necrotic factor-α,
a cortisol level sensor for determining the cortisol level of the person,
a C-reactive protein sensor for determining C-reactive protein data related to the person's C-reactive protein,
a melatonin sensor for determining melatonin data related to the person's melatonin concentration, and
a device as disclosed herein for managing treatment of an inflammatory autoimmune disease of a person based on the obtained measurement data acquired over time by the white blood cell counter and said sensors.

In yet further aspects of the present invention, there are provided a computer program product which comprises program code means for causing a computer to perform the steps of the method disclosed herein when said computer program is carried out on a computer as well as a non-transitory computer-readable recording medium that stores therein, which, when executed by a processor, causes the method disclosed herein to be performed. The computer program product can comprise computer-readable program code downloadable from or downloaded from a communications network, or storable on, or stored on a computer-readable storage medium, which computer-readable program code, when run on a computer or processing unit causes the computer or processing unit to perform the steps of any embodiments of the method in accordance with the present invention. The computer program product can be suitable to work with a system including a server device and a client device. Part of the steps can be performed on the server device while the other or another part of the steps is performed on the client device. The server and client device can be remote from each other and connected through wired or wireless communication as known in the art. Alternatively, all steps are performed on a server device or on a client device. The server device and client device can have communication devices for communicating with each other using wired or wireless communication protocols.

Preferred embodiments of the invention are defined in the dependent claims. It shall be understood that the claimed method, system, computer program and medium have similar and/or identical preferred embodiments as the claimed device and as defined in the dependent claims.

Similar to the majority of the cases drug intake for medications used in RA do not follow any specific criteria concerning the time of the day of the administration (in a 24 hours cycle). It is rather administered based on the normal subdivision of the 24 hour day to morning, lunch, afternoon, dinner and before bedtime. The emphasis is placed on the interval of time in between the intake of the medication (number of hours)

Moreover, drug administration does generally not take into account any particularity of the patient's routine such as specific life rhythms (normal bed time or wakeup time, number of working hours, exercise patterns, seasonality, etc.). These aspects of patient's life contribute enormously to the circadian rhythm and therefore are highly relevant to the management of the disease.

Synchronizing these “personal” aspects of patient's routine with drug administration frequency, period and possibly dosage or, more generally, managing treatment of the inflammatory autoimmune disease of the person based on the obtained measurement data over time by the input unit, will provide the patients with method to optimize the treatment and management of disease leading to patient empowerment and acting as an enabler for the patient to better adapt to the effect of the disease and minimize the impact of the disease symptoms on their daily routine.

Additionally, the proposed approach will reduce the negative psycho-social effect of RA and can therefore lead to reducing fatigue and depression enhancing the patient's quality of life dramatically.

Conventionally, in contrast, the management of the disease is done by the physician during the periodic visits, based on laboratory test (at the time of the visit), questionnaires and observations. Between the visits, patients and/or clinicians cannot track the disease progression and/or response to the therapy prescribed. Patients also do not have tools that can help them to adapt the administration of the drugs to their specific needs (day/night rhythm, activity level, etc.). The proposed approach will allow a personalized treatment management, reducing the negative impact on patients' lives. Patients will have more control of the disease as well as the treatment side effects, which will help them to adapt to their new situation faster and more effectively.

At least the five main parameters including the person's white blood cell count, the person's tumor necrotic factor-α, the person's C-reactive protein, the person's cortisol level and the person's melatonin concentration are used to objectively assess the treatment of the inflammatory autoimmune disease of the person, in particular while the person is undergoing a therapy. These measurement data are generally acquired over time, i.e. the measurement data are collected using several point measurements during a period of time, i.e. from time to time (e.g. at regular or irregular intervals). Alternatively, continuous measurements may be made, if available or possible (however, such a continuous measurement is not generally required). Based on such an objective assessment the person's treatment can be adapted. Further, it enables more effective timing of treatment interventions and, consequently, increases the patient's well-being. Still further, continuous or semi-continuous monitoring of the person is possible, accounting for both short term (i.e., hour to hour, day to day) and long term (week to week, month to month) fluctuations and variations in treatment parameters.

According to the invention said treatment manager is configured to determine a disease activity level and to monitor the disease activity level over time. This enables an early recognition if the treatment is successful. The disease activity may e.g. be determined by providing a score for each parameter used in the management of treatment and for commonly evaluating the different scores to obtain a combined score reflecting or representing the disease level. The obtained measurement data and/or other available data and/or scores may be used for determining the disease activity level.

Using at least the proposed parameters a person can follow his status after every therapy session with the aim to predict, recognize and hence relieve the disease symptoms. This approach can also detect any abnormality in the recovery progress resulting in a significant reduction of the negative impact of the treatment on person's life.

In an embodiment said treatment manager is configured to monitor trends over time in at least one, preferably all, of the obtained measurement data by the input unit. By monitoring the trends early detection of negative effects or of potential improvements of the treatment is possible.

In another embodiment said treatment manager is configured to combine the obtained measurement data by an algorithm, in particular based on a base line value of one or more parameters for the person (i.e. one or more of the above mentioned parameters corresponding to the measurement data) as well as monitoring and analyzing their trend during treatment time, and to use the combined measurement data, alone or in addition to one or more of the obtained measurement data, for managing treatment For instance, a score or a result of a function may be calculated, as “combined measurement data”, from some or all of the obtained measurement data, which is then used to consider changes of the treatment.

In another embodiment the device further comprises an interface for issuing treatment information, user information, therapy recommendations and/or decision support. Treatment information may include information if treatment is successful and if there are any negative effects on the patient's health condition, e.g. if there are any unexpected or expected side effects of the therapy. User information may include information, e.g. for a doctor, informing the user about the result of the treatment, e.g. about a level of treatment success as detected over time. Therapy recommendations may include recommendations for the person and/or a user how the therapy should be continued or modified or which other therapies should be applied. Decision support may include information e.g. for a doctor supporting him to make decisions with respect to the person, e.g. how to continue with the therapy.

In an embodiment the input unit is further configured to obtain temperature data and/or cytokine data of the person acquired over time (i.e. from time to time or continuously) and wherein said treatment manager is configured to additionally use the obtained temperature data and/or cytokine data for managing treatment. This further improves the quality of the treatment management.

Preferably, said treatment manager is configured to additionally extract chronobiology information from one or more parameters related to the chronobiology of the person for managing treatment, the treatment manager being further configured for managing treatment based on said extracted chronobiology information. It has been found that parameters of the person's chronobiology are related to treatment of a disease. Monitoring of the five above mentioned parameters over time can provide the required information on the patient-specific chronobiology aspects of disease treatment. Other parameters include sleep disorder related parameters such as cortisol, melatonin including their relationship, e.g. ratio, activity during sleep, brain function data, muscle activity and/or fatigue level parameters, heart rate and heart rate variability, respiratory function level, temperature. These parameters can be measured by various (additional) sensors or can be collected by available devices used by the person, such as a smartphone, smart watch, smart patch, a camera, etc. Thus, the use of chronobiology information and the link with treatment results further improves the correct, early and objective management of treatment. Further, the disease status of the person may be monitored.

In still another embodiment said input unit is configured to obtain person activity data (also called “soft data” herein) related to one or more activities of the person, wherein said treatment manager is configured to additionally use the obtained person activity data for managing treatment. Said person activity data may include one or more of diet or eating habits, exercise frequency, activity level, and sleep disturbance (e.g., through activity based e.g. through accelerometers for detecting motion during sleep at night, or brain signal e.g. delta wave measurement, etc.). The proposed system thus may comprise additional corresponding sensors or means for acquiring the respective data. These sensors might be ‘embedded’ or otherwise connected (wired, wireless, via the cloud, etc.) to the system.

The input unit may also be configured to obtain physiological data (also called “hard data” herein) related to one or more physiological parameters of the person, wherein said treatment manager is configured to additionally use the obtained physiological data for managing treatment. Said physiological data may include biomarker data from blood. Also for acquisition of the respective data the proposed system may comprise corresponding sensors or means. Thus, in these embodiments the problems of the known methods and devices are solved by multi-component monitoring of various parameters related to treatment of a disease.

In still another embodiment said treatment manager is configured to determine for the obtained data a respective deviation of the respective measurement data from a predetermined range, in particular a person-related range, for combining, in particular adding, said deviations and for managing treatment, in particular for scheduling the timing of treatment, based on the combined deviations. The combination may be obtained by various multi-criteria decision analysis techniques, e.g., weighted summation, weighted product model, aggregated indices randomization method, etc.

The proposed system may additionally comprise, besides the above mentioned sensors, one or more of a video camera, a microphone, a body wearable sensor and a stationary sensor for obtaining (or receiving) person activity related to one or more activities of the person and/or physiological data related to one or more physiological parameters of the person.

The proposed approach, using at least the same mandatory parameters can be used for various other inflammatory autoimmune diseases apart from RA, like: psoriasis, systemic lupus erythematosus, inflammatory bowel disease, inflammatory myopathies as well as asthma. Preferably, the parameters are adjusted to make the system more specific for each different disease.

The steps of the proposed method may be performed on a stationary or mobile apparatus, e.g. an apparatus which may be worn by the person or which may be used by a physician for visits of patients. For instance, a user device such as a smart phone, a mobile phone, a laptop, a computer, a tablet, a watch, a camera, may be used. Preferably, the steps of said method are then implemented in software or an application program (“app”), performed e.g. on a processor or computer.

An example of the device for managing treatment of an inflammatory autoimmune disease of a person according to the present invention may comprise an input unit for receiving measurement data acquired over time including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration, and a treatment manager for managing treatment of the inflammatory autoimmune disease of the person based on the obtained measurement data.

It will be appreciated by those skilled in the art that two or more of the above-mentioned options, implementations, and/or aspects of the invention may be combined in any way deemed useful.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system and a device according to the present invention,

FIG. 2 shows a schematic diagram of a second embodiment of a system and a device according to the present invention,

FIG. 3 shows a schematic diagram of a third embodiment of a system and a device according to the present invention,

FIG. 4 shows a diagram illustrating the chronobiology,

FIG. 5 shows a diagram illustrating the effects of sleep and loss of sleep on the circadian production of the nocturnal hormones melatonin and cortisol,

FIG. 6 shows a diagram illustrating the circadian rhythm of a patient, and

FIGS. 7 to 12 show graphs of the behavior of various parameters over time.

### DETAILED DESCRIPTION OF THE INVENTION

FIG. 1 shows a schematic diagram of a first embodiment of a system 1 and a device 2 according to the present invention for managing treatment of an inflammatory autoimmune disease of a person. Besides the device 2, the system 1 comprises a white blood cell counter 3 for counting the white blood cells of the person, a tumor necrotic factor-α sensor 4 for determining the tumor necrotic factor-α data related to the person's tumor necrotic factor-α, a cortisol level sensor 5 for determining the cortisol level of the person, a C-reactive protein sensor 6 for determining C-reactive protein data related to the person's C-reactive protein, and a melatonin sensor 7 for determining melatonin data related to the person's melatonin concentration. Based on the obtained measurement data acquired over time by said sensors 3 to 7 the device 2 manages treatment of an inflammatory autoimmune disease of the person. Further, the effects of the treatment may be monitored over time.

In treatment of chronic diseases such as RA, where a cure to remove the root cause of the disease does not exist, the accepted clinical approach consists of alleviating and/or diminishing the effect of the disease symptoms (e.g. pain) as well as trying to slow down the progression of the disease. This approach is known as “disease management” or “treatment management”.

The device 2 comprises an input unit 20 for receiving (or obtaining) measurement data acquired over time including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration. A treatment manager 21 manages treatment of the inflammatory autoimmune disease of the person based on the obtained measurement data. The input data may be any kind of data interface which directly obtains the measurement data from the respective sensor (i.e. the sensors 3 to 7, and optionally additional sensors, of the system 1), e.g. through a wireless or wired connection, to process the data on the fly and immediately detect if the person's treatment is successful or if any changes should be made, how the disease progresses and/or to which response a treatment leads. Alternatively, the data may be stored or buffered, e.g. in a storage medium, a hospital's data base, etc. for later processing by the device 2.

The treatment manager 21 may be a processor of a separate device or a computer that is particularly programmed for carrying out the analysis.

The white blood cell counter 3 may be a sensor that counts blood cells in a blood probe taken from the person using existing methods suitable for ex-vivo blood analysis. Alternatively, a variety of non-invasive blood counts may be performed, the majority of which allows in-vivo testing. These methods include electrical approaches (as e.g. described in Electrical admittance cuff for noninvasive and simultaneous measurement of haematocrit, arterial pressure and elasticity using volume-oscillometric method, Yamakoshi K1, Tanaka S, Shimazu H., Med.Biol>Eng. Comput. 1994 July; 32(4 Suppl):S99-107) or ultrasound approaches (as e.g. described in Noninvasive in vivo measurements of hematocrit. Secomski W et al., J. Ultrasound Med. 2003 April; 22(4):375-84) as well as a variety of known optical measurements of the white blood or red blood cell counting methods with or without labeling techniques (as e.g. described in Direct measurement of microvessel hematocrit, red cell flux, velocity, and transit time, Sarelius I H, Duling B R. Am J. Physiol, 1982, December; 243(6):H1018-26 and Noninvasive imaging of flowing blood cells using label free spectrally encoded flow cytometry, Lior Golan et al., Biomed Opt Express, 2012, Jun. 1, 3(6) 1455-1464).

The tumor necrotic factor-α sensor 4 may be a sensor that determines the tumor necrotic factor-α data related to the person's tumor necrotic factor-α. The C-reactive protein sensor 6 may be a sensor that determines C-reactive protein data related to the person's C-reactive protein. The melatonin sensor 7 may be a sensor that determines melatonin data related to the person's melatonin concentration. Generally, all these parameters are obtained by blood based measurements. Hence, the sensors 4, 6 and 7 are generally blood based sensors, such as separate immunoassays (or a common immunoassay

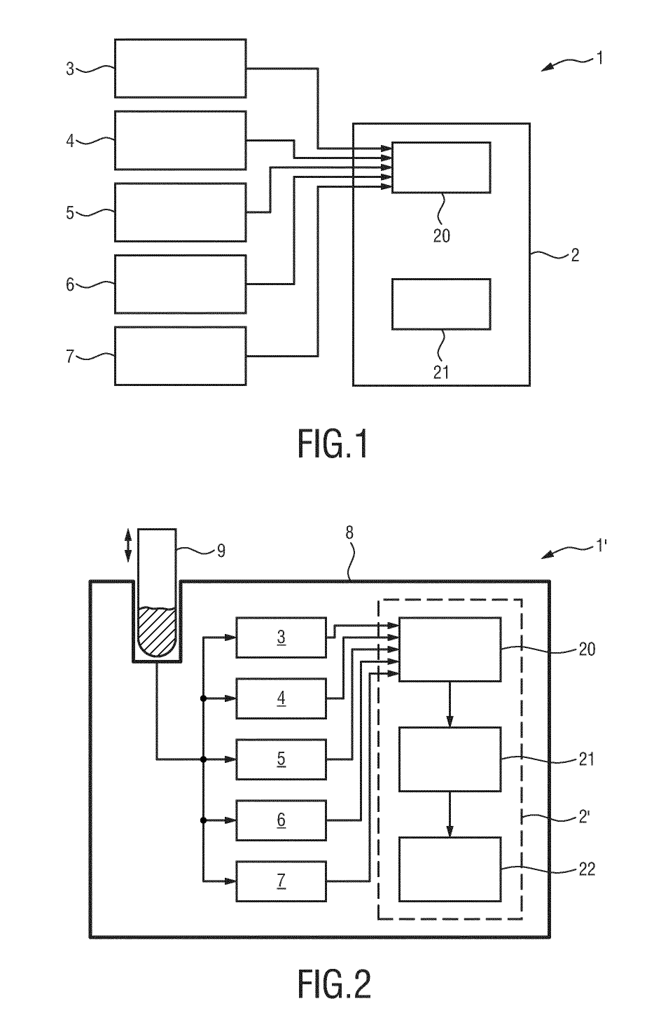

The system 1 may be used for managing treatment of an inflammatory autoimmune disease of a person from time to time or regularly or even continuously, e.g. to monitor trends over time. For this purpose, the treatment manager 21 may be configured to monitor trends over time in at least one, preferably all, of the obtained measurement data. FIG. 2 shows a schematic diagram of a second embodiment of a system 1′ and a device 2′ according to the present invention. In this embodiment all elements of the system 1′ are integrated into a single apparatus 8, which may be a stationary or mobile apparatus, e.g. an apparatus which may be worn by the person or which may be used by a physician for visits of patients.

In an embodiment the apparatus 8 may be of the same or similar type as the device for home monitoring of hematological parameters of patients as described in WO 2014/024176 A1 or of the same or similar type as the commercial device Minicare H-2000, which is a remote monitoring system for patients undergoing chemotherapy. One or more probes 9 of a body fluid, particularly blood, are used for acquiring some or all of the measurement data described above. For this purpose the respective appropriate sensors 3 to 7 are incorporated into the apparatus 8 so that the person can perform a self-diagnosis.

The device 2′ further comprises an interface 22 for issuing treatment information, user information, therapy recommendations and/or decision support. Treatment information may include the determined response to treatment and/or a progression of the disease, a trend of the response and/or progression over time, or information if treatment and/or progression is proceeding as expected or not. User information may include information how and/or when to use the system 1′, or information if and how the treatment proceeds, if and how the disease progresses. Therapy recommendations may include recommendations if and how the therapy of the person shall be continued, changed or stopped, e.g. if a chemotherapy shall be modified. Decision support may include information directed to a physician about the response to the treatment and/or the disease progression and supporting the physician to make a decision if and how the therapy shall be modified.

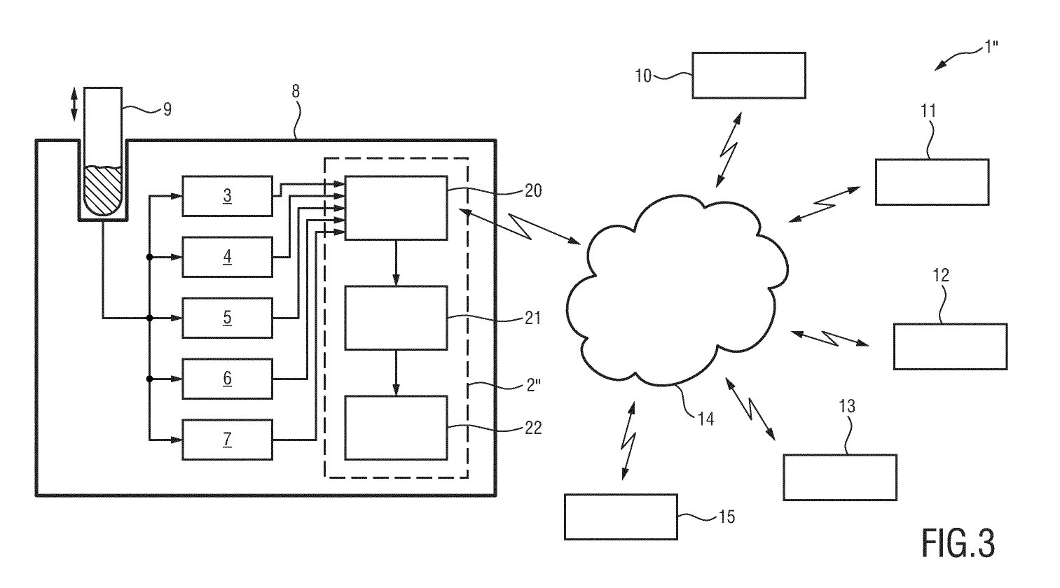

FIG. 3 shows a schematic diagram of a third embodiment of a system 1″ and a device 2″ according to the present invention. The device 2″ is preferably configured in the same way as the device 2′ shown in FIG. 2 and is incorporated into an apparatus 8 together with sensors 3 to 7. In another embodiment, however, the device 2″ is configured in the same way as the device 2 shown in FIG. 1 with external sensors 3 to 7. In still another embodiment the device 2″ is configured by combining elements of the devices 2 and 2′, i.e. where part of the sensors 3 to 7 are included in the apparatus 6 and the other part of the sensors are provided as external sensors.

The system 1″ employs a multi-component approach, combining “soft” data, which are particularly person activity data related to one or more activities of the person, and/or “hard” data, which are particularly physiological data related to one or more physiological parameters of the person, that are relevant to the treatment and the treated disease. The input unit 20 of the device 2″ is thus configured to obtain such person activity data and/or physiological data, and the treatment manager 21 is configured to additionally use the obtained person activity data and/or physiological data for treatment management. For instance, a semi-continuous assessment of the treatment and/or disease state of an RA patient can thus be realized.

The person activity data may include one or more of eating habits, exercise frequency, activity level, sleep disturbance, speech pattern, eye movement and body posture, and the physiological data may include biomarker data from blood. To obtain such data additional sensors are used, as shown in FIG. 3, including one or more of a microphone 10, a video camera 11, other stationary and/or wearable sensors 12 (e.g. a vital signs camera, smart bed, smart chair, etc.) and wearable devices 13 (e.g. smart watches, smart phones, Google Glass, smart patches, electrode skull caps, etc.).

The wearable and non-wearable devices can be linked to device 2″ directly, via a wired or wireless network (e.g. a WiFi network), via the cloud 14, or simply via a telehub component. It may thus also be possible to control one or more of the various sensors 3 to 7 and 10 to 13 when, how long and how often measurement data shall be acquired and provided to the device 2″.

A database 15 containing the patient's history as well as data from prior therapy cycles or from before starting the therapy may also be linked to the device 2″, e.g. also via the cloud 14. A disease score may be used in combination with the database 15 to manage the patient's therapy by devising personalized exercise routines, nutritional advice and relaxation therapy. In addition, the disease score can be used by the physician to help in the scheduling of the next round of therapy and to advise on hospital admission.

More specifically, the “soft” data (person activity data) can be obtained using the proposed device, preferably with an additional microphone and video camera, as well as with other wearable and/non-wearable devices, in the following ways:
1. Cortisol level—Cortisol is a steroid hormone released by the adrenal gland metabolic which triggers mechanisms leading to production of compounds used as energy sources in emergency conditions. Cortisol is a validated marker for stress. An increased blood cortisol concentration (of up to 64%) has been reported in all chemotherapy patients. Moreover, there is a link between endogenous cortisol level in predicting acute and delayed nausea during chemotherapy. Using a built-in blood analysis apparatus of the device it is possible to measure the cortisol blood concentration level.
2. Melatonin level—Melatonin is a hormone, produced by the pineal gland, which regulates the body's sleep-wake cycle. Melatonin levels fluctuate throughout the day. Using a built-in blood analysis apparatus of the device it is possible to measure the melatonin blood plasma concentration level.
3. Galvanic skin response (GSR)—Increased GSR is associated with increased stress and fatigue. This can be measured using skin electrodes in a smart watch or other wearable device.
4. Resting Heart Rate (HR)—Elevated HR may be associated with anemia. HR can be measured using various contactless and contact methods, including a vital signs camera, as well as green photoplethysmogram (PPG) and accelerometer sensors integrated in a smart watch or smart patch.
5. Resting Heart Variability (HRV) index—Decreased HRV is linked to increased fatigue. HRV can be measured using various methods, including a vital signs camera, as well as green PPG and accelerometer sensors integrated in a smart watch or smart patch.
6. Red blood cell (RBC) count—Low red blood cell count is associated with anemia. Using a built-in blood analysis apparatus of the device it is possible to determine the RBC count whenever the patient analyzes their blood.
7. Skin temperature—Skin temperature increases with increasing fatigue level due thermal dysregulation (fever). This can be measured using a temperature sensor integrated in a smart watch worn around the patient's wrist.
8. Systolic blood pressure (BP)—Systolic BP increases with increasing stress and fatigue. This can be measured using the pulse arrival time obtained with the green PPG sensor in the smart watch. It can also be obtained from electrocardiogram (ECG) or BP cuff measurements.

Based on the obtained disease score or other information related to the disease progression and/or response to treatment, appropriate and personalized clinical intervention can be undertaken to improve the management of treatment and prevent unnecessary hospitalization. This may involve a home visit by a nurse or general practitioner, scheduling of an outpatient visit and hospitalization (if required). In addition, the obtained information may be used to assist in determining whether patients are ready for their next round of therapy and to detect trends which are useful for forecasting. Additionally, based on the obtained measurement data, a personalized program can be devised to support the patient in various ways, including nutrition advice (including appropriate supplements), relaxation routines and/or targeted exercise routines aimed at fighting fatigue, nausea, muscle mass reduction, bone density reduction, depression.

In still another embodiment the treatment manager 21 is configured to additionally extract chronobiology information from one or more parameters related to the chronobiology of the person for managing treatment. The circadian pattern of various cytokines and hormones in RA disease activity provides an opportunity for introducing a new treatment paradigm. The clock for RA allows patients to know and follow their chronobiology in order to enhance the efficacy of the therapy prescribed. By taking into account the day-night patterns the RA patients can benefit from chronotherapy, an approach that includes timing medications to 24-hour rhythms in disease pathophysiology in order to optimize the result of the medication.

Similar to several other conditions in general, but specifically as an autoimmune disease, the response of the RA patients to identical medications is subject to person-to-person as well as chronobiological variations. Chronobiology is a field of biology that examines periodic (cyclic) phenomena in living organisms and their adaptation to solar- and lunar-related rhythms. These cycles are known as biological rhythms. By taking into account the chronobiology of the patient in disease management we mean gathering the relevant data during 24 hour cycles in order to establish the patient specific circadian rhythm as well as the effect of this rhythm on the immune system and disease biomarkers.

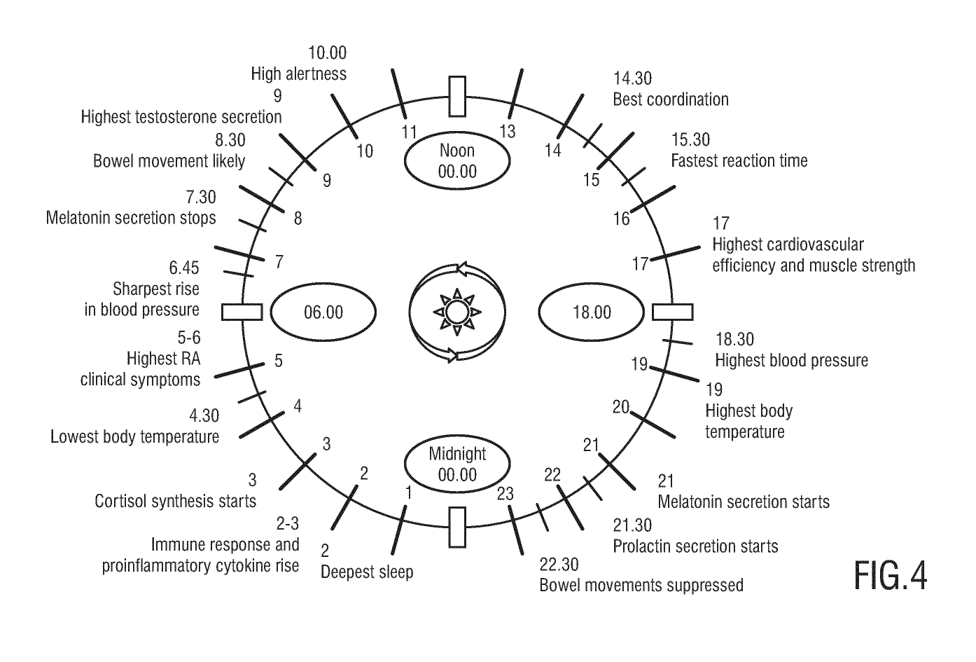

FIG. 4 shows a diagram illustrating that the chronobiology of the most important physiological activities (i.e. cardiovascular and muscle function, sleep) and altered neuroendocrino immune-related activities and clinical symptoms in rheumatoid arthritis (RA) are based on a cyclical 24-h period.

Hence, in an embodiment chronotherapy is used which means scheduling the timing of medical interventions according to biological rhythm determinants as a means to optimize treatment outcomes and minimize or avoid adverse effects. This approach will help patients to take control of their disease faster allowing them to adapt to their new condition of living with RA.

Evening scheduling of methotrexate in synchrony with rise in the cytokine biomarker TNF-α significantly enhances its effectiveness. Additionally, it has been proven that timing of glucocorticosteroids (GC) therapy to its nocturnal rise better reduces morning RA symptoms and functional disability compared to either once-daily morning or bedtime conventional prednisone tablet therapy.

The efficacy of chonotherapy is may thus be efficiently used in everyday practice. To accurately determine the circadian rhyme of the patient a personalized chronotherapy clock may be used. With this clock an algorithm may be used by combining specific biomarkers for the circadian rhythm such as cortisol, melatonine and prolactin together with temperature and cytokines to determine and predicted the chronobiology of the patient and suggest the best time to take medication.

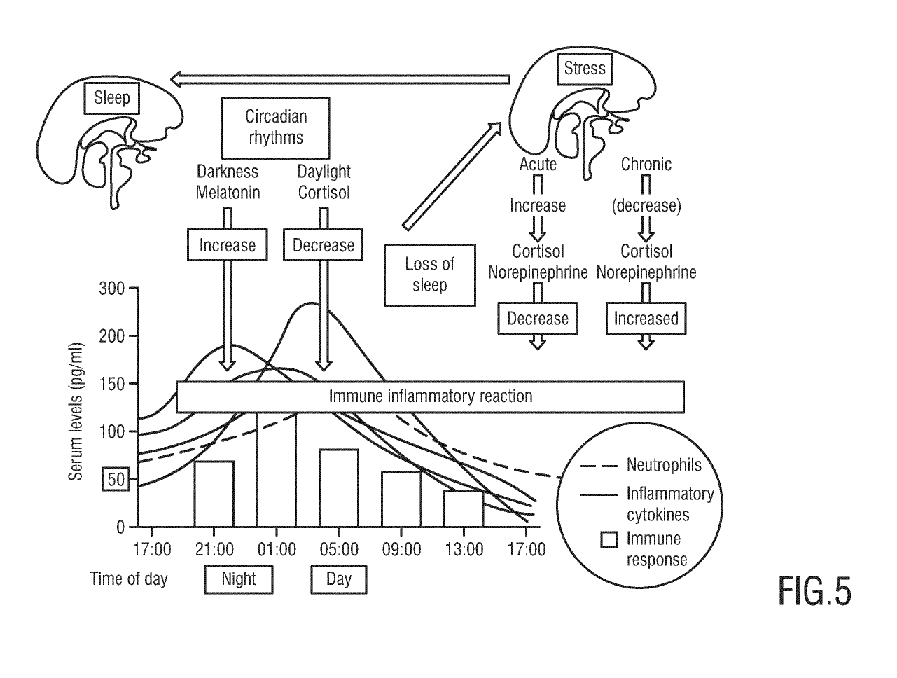

FIG. 5 shows a diagram illustrating the effects of sleep and loss of sleep on the circadian production of the nocturnal hormones melatonin and cortisol. Both hormones modulate the immune/inflammatory reaction during the night (up-regulation and down-regulation, respectively) and in chronic RA are unbalanced because of a decrease in the endogenous cortisol (immune suppressive and antiinflammatory hormone) and an increased efficacy of melatonin (and prolactin) acting as immune response enhancers.

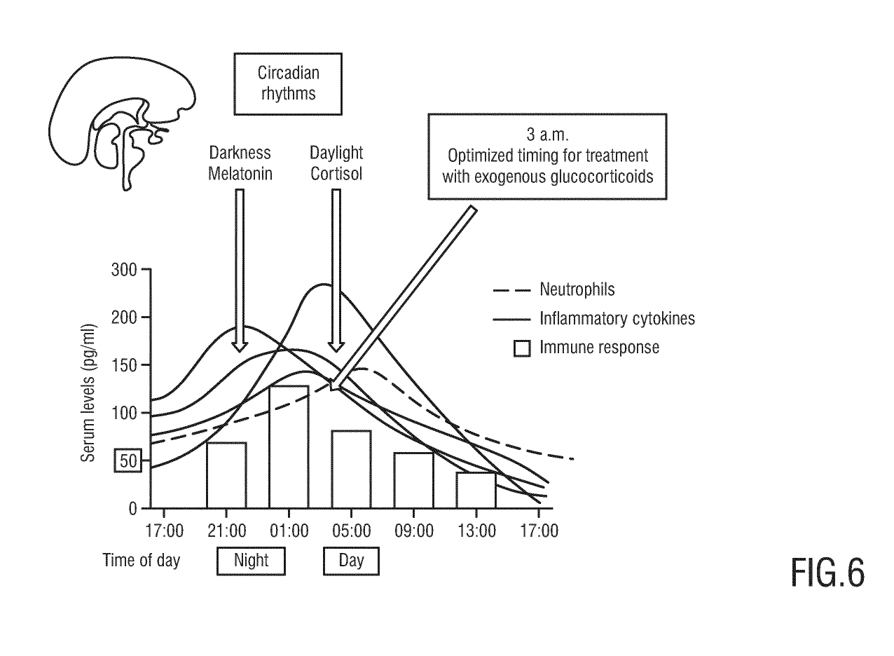

FIG. 6 shows a diagram illustrating the circadian rhythm of a patient. To improve the risk-benefit ratio of glucocorticoids in RA treatment is the use of low-dose prednisone chronotherapy with modified-release prednisone, a timing drug release (releasing prednisone at 3 a.m.) to modulate the chronobiological rhythms of inflammation.

This personalized RA clock for patients may be based on objective measurements of a number of several key parameters which are highly specific to chronobiology, because of they are related to the immune and metabolic systems of the body. In a particular implementation an apparatus of the type as shown in FIG. 2 may be used for obtaining these parameters.

In an embodiment the following parameters may be used:
1. WBC (White Blood Cell) count
2. TNF-α (Tumor necrotic factor-α), IL-6 (Interleukin 6), CRP (C-Reactive Protein)
3. HG (hemoglobin), red blood cell count related to anemia
4. T (Temperature)
5. ΔP (changes in Prolactin)
6. ΔC (change in cortisol concentration)
7. ΔM (change in melatonin concentration)

An exemplary algorithm for processing these parameters may be:

DA(t)=F{ρ((a*WBC+g*Cytokines)+(b*HG)+(c*T)+(d*AP)+(e*ΔC)+(f*ΔM))}

wherein DA(t) is the disease activity as a function of time. An algorithm may be used to develop a drug administration routine using the DA(t) function as input. The coefficients a-g represent the “weight” of each parameter. The time t=[0, 24] refers to time in a 24 h cycle. The “normal” range for each parameter is known through clinical data.

It should be noted that the algorithm to combine several weighted parameters can also take the format of parameters that are in various expressions (for example the inverse of one or square root or log of the other). This will be apparent after doing the trend analysis. In the above example the general form of the algorithm has been presented, which can include the alternatives mentioned above.

Cortisol and melatonin are interrelated validated biomarkers the fluctuation of which during a 24 hour period indicates the health of the HPA axis with significant influence on pro-inflammatory cytokines synthesis and sleep quality. Clinically accepted threshold levels for each parameter have been (or may be) created through clinical practice for these parameters. Statistical analysis of such existing data for healthy population provides trends for minimum, maximum, and acceptable fluctuations in these parameters. By combining the acceptable threshold for healthy population with the data trends obtained via analysis of the parameters for the RA patient the “weight” of the parameter represented by coefficients in the formula shown above can be determined.

The parameters selected for management of treatment are particularly important in the determination of the chronobiology of the RA for each patient as people are not identical in this regard and hence do not respond to therapeutics in an identical way. This information is then applied to the management of the disease and making treatment personalized.

For instance, in an RA patient the inflammatory parameters like WBC (White Blood Cell) count, TNF-α (Tumor necrotic factor-α) and CRP (C-Reactive Protein) have higher concentrations than in a healthy individual at a given time because RA is by definition an inflammatory disease while the concentration trend for each parameter is also influenced by chrono-behavior. The treatment routine aims to manage the disease by establishing the normal trend in these parameters.

According to the present invention the chronobiology may thus be taken into account in protocols for drug administration resulting in a match between the time of drugs intake and the time when the fluctuations of the inflammatory parameter appear.

In an embodiment, initially the used algorithm is used to create a baseline for which multiple values are needed to determine the chrono-biological trends. Drug administration will change these trends. It is therefore proposed to monitor their fluctuation in order to determine the best routine for drug administration. Typically, symptoms of a disease like RA are worst in the morning and a number of medications are taken once daily. The baseline and subsequent measurements are thus made to determine the best time (and dose) for the medications for optimal management of the disease. This will lead to reduced impact of the disease. The specific algorithm may be developed or adjusted to the particular patient and disease by trend analysis for various parameters in comparison with normal range for each parameter.

A platform like the above described and known Minicare platform may be used with minimally invasive collections of blood (e.g. by finger prick). For the calculation of the circadian rhythm and for establishing the baseline, a minimum of 3 sample collections may be needed per day during the first days, e.g. the first 3 to 4 days. After the baseline is established and the nature of fluctuations determined only one sample collection may be sufficient per day.

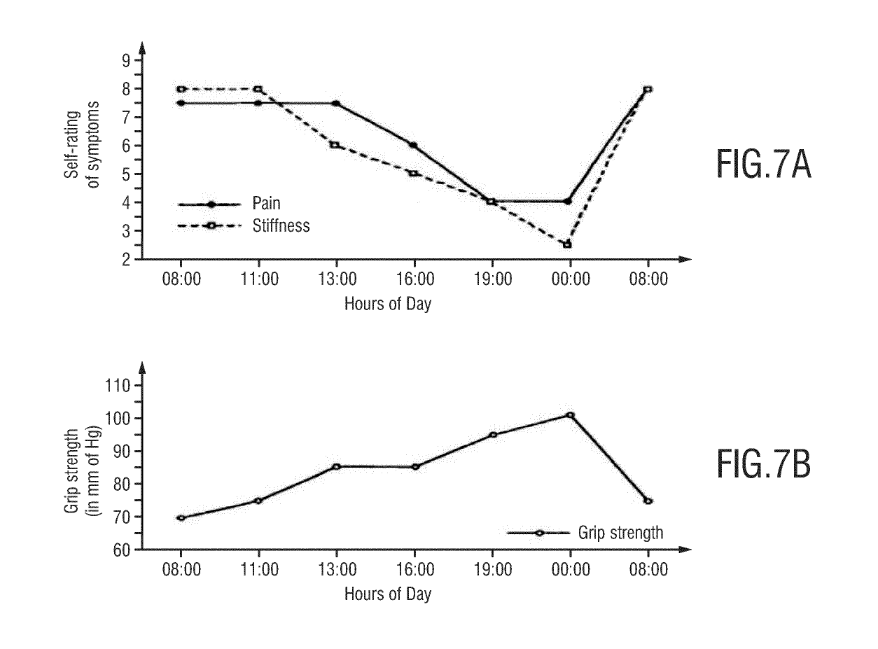

FIGS. 7 to 12 show graphs of the behavior of various parameters over time.

FIG. 7 illustrates circadian variations in signs and symptoms in patients with RA. FIG. 7A shows the self-rating of symptoms and FIG. 7B shows the grip strength. Data were obtained from one patient with RA conducting self-assessment of pain, stiffness and grip strength several times daily while taking 100 mg Flurbi at 0900 and 2100 hrs.

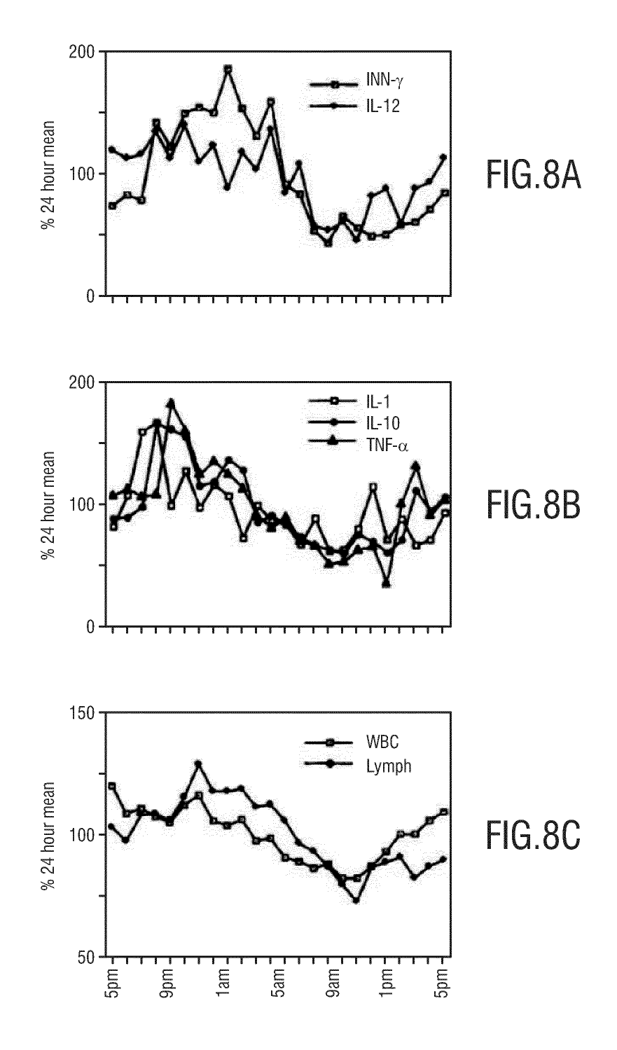

FIG. 8 illustrates diurnal rhythms of cytokines and white blood cells in healthy individuals. FIG. 8A shows the LPS-stimulated whole blood production of IFN7 and IL-12. FIG. 8B shows the LPS-stimulated whole blood production of IL-1, 11-10 and TNF-α. FIG. 8C shows the white blood cell (WBC) and lymphocyte (lymph) count in whole blood. The results shown are the means of individual subject data (n=10) standardized as a percentage of each subject's 24-hour mean.

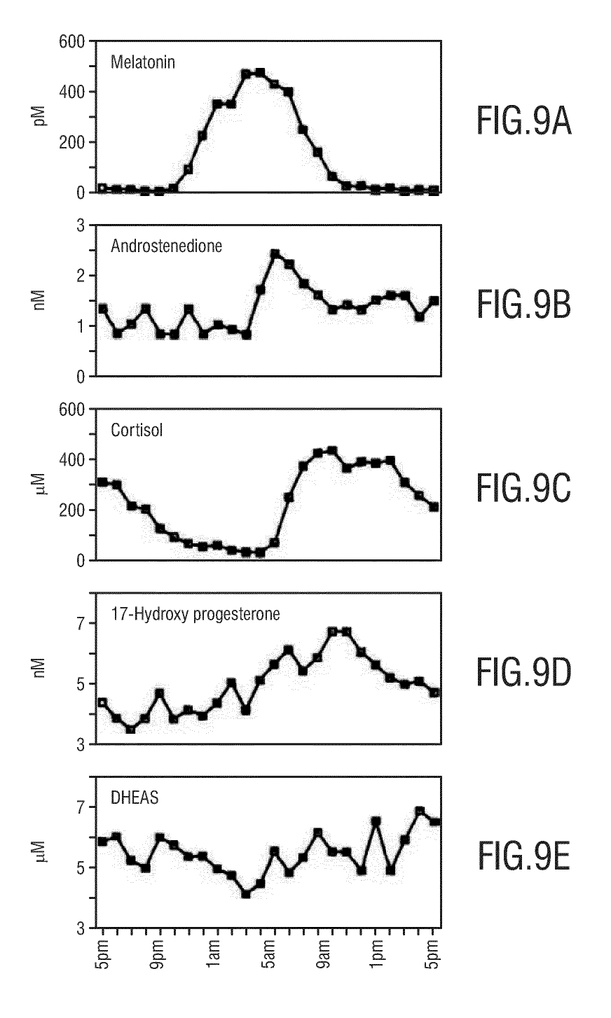

FIG. 9 illustrates the diurnal rhythms of plasma melatonin (FIG. 9A), androstenedione (FIG. 9B), cortisol (FIG. 9C), 17-hydroxyprogesterone (FIG. 9D) and DHEAS (FIG. 9E). Results shown are the data of one representative subject.

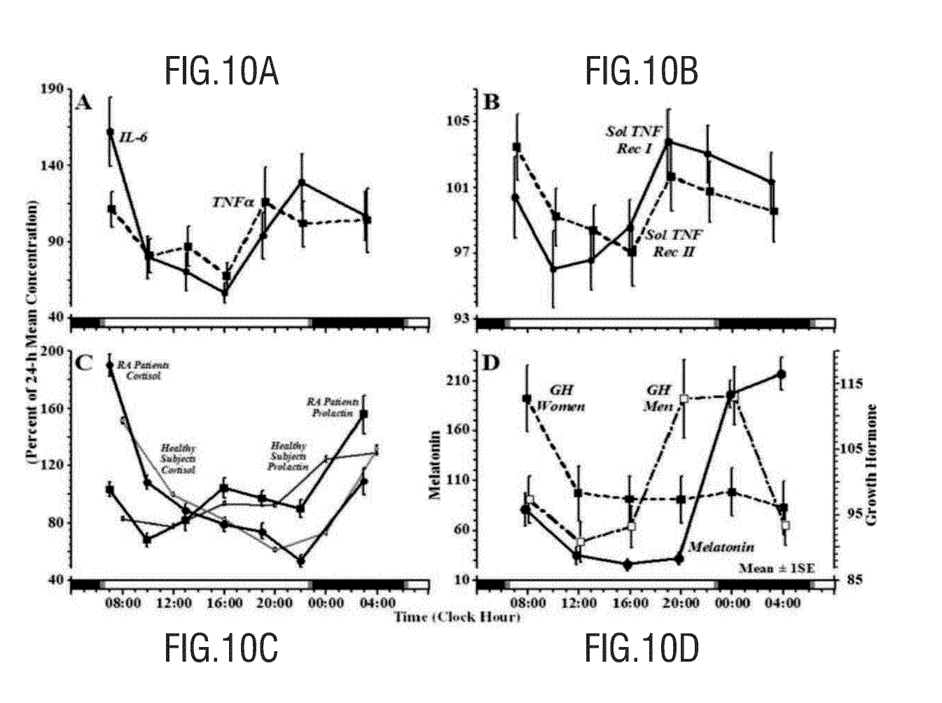

FIG. 10 illustrates the circadian variation of TNFα and IL-6 (FIG. 10A), soluble TNF receptors I and II (FIG. 10B), cortisol and prolactin in 29 RA patients with active disease (FIG. 10C), and prolactin and cortisol in 281 and 331 clinically healthy adults, respectively, and pro-inflammatory acting melatonin in 20 clinically healthy subjects and of growth hormone in 309 clinically healthy adults (FIG. 10D). All results are presented as percent of the 24-hour mean concentration of each variable to eliminate inter-individual differences. Actual cortisol 24-hour means (±S.E.) for the 331 healthy subjects and 29 RA patients are 9.4±0.2 μg/dl and 10.4±0.6 μg/dl, respectively; prolactin 24-hour mean (±S.E.) is higher in the 280 clinically healthy subjects (20.7±0.7) versus the 29 RA patients (8.6±0.5 ng/ml); GH 24-hour means (±S.E.) for the 142 healthy adult men and 167 women are 1.70±0.09 ng/ml and 1.98±0.1 ng/ml, respectively. Black and white shading along the bottom time axis indicates, respectively, the subjects' nighttime sleep and daytime activity. Significance of the difference in absolute values will require further research with matched populations.

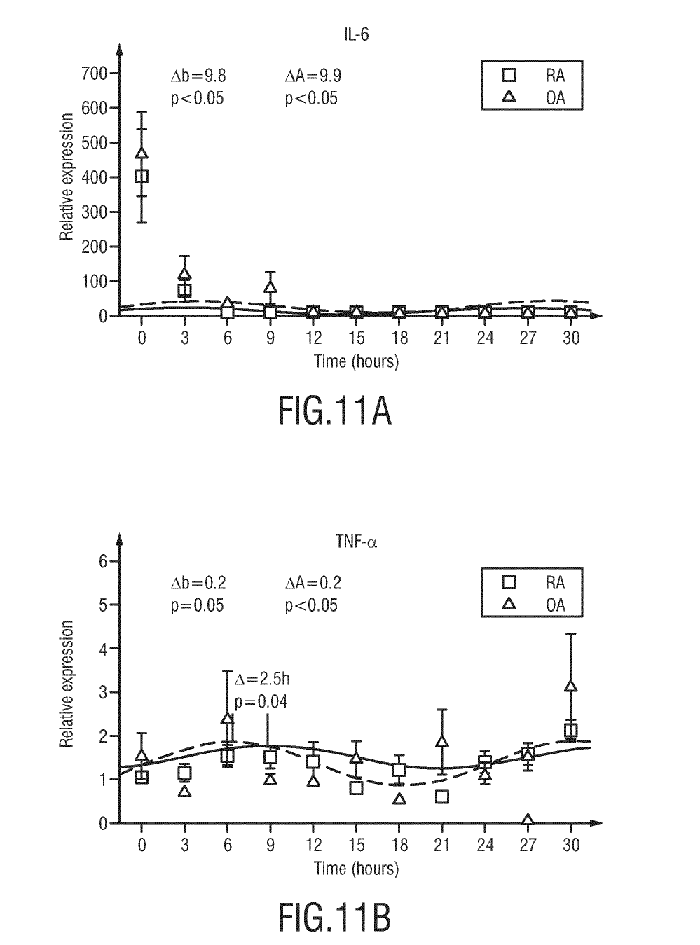

FIG. 11 illustrates the oscillation of IL-6 (FIG. 11A) and TNF-α (FIG. 11B) in RA and OA cells after serum shock. Relative gene expression over time is displayed. Fibroblasts derived from RA synovium display weaker rhythmic expression of IL-6 and TNF-α after clock resetting than cells from OA synovium. Fitted sin graphs of the expression and mean 6 SD of the mean from three individual patients per group are shown. The time of serum removal is 0 in the graphs and the relative expression is normalized to the expression before serum (at time=22). Δb indicates the general difference in the expression i.e., the baseline of the expression. ΔA indicates the differences in the peak amplitude. Phase differences a are marked as red solid lines and the difference is indicated in hours.

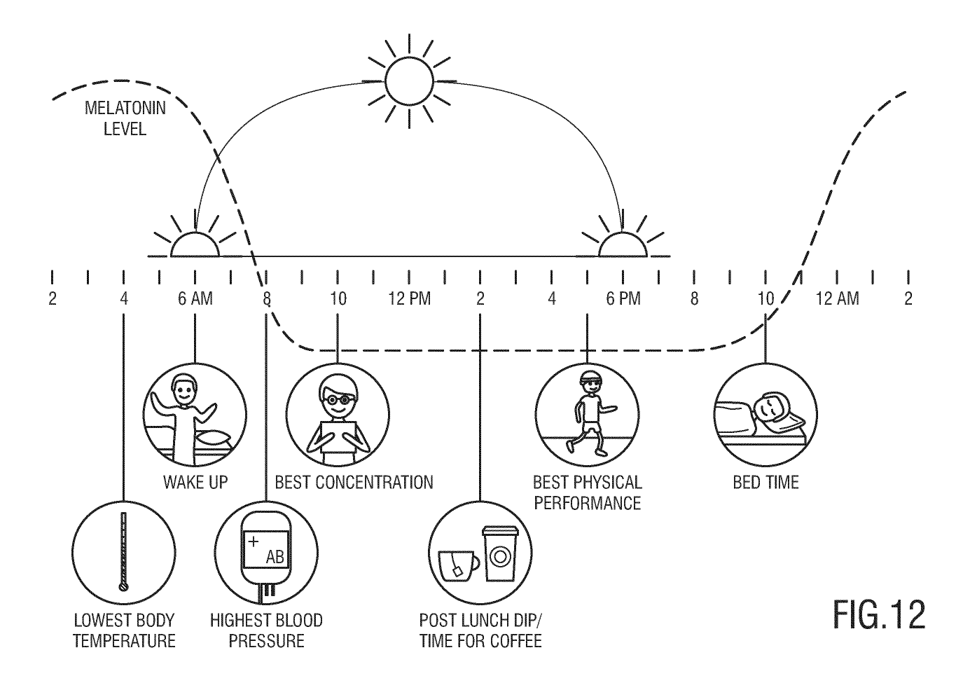

FIG. 12 shows the course of the melatonin level over the course of a day.

In an exemplary implementation a typical disease management scenario may be as follows: Person X is under treatment for an autoimmune disease (e.g. RA). The effect of the treatment that patient X is receiving is measured and monitored. A set of parameters as explained above are used to do this monitoring through determining how these parameters behave in reaction to the treatment. Chrono-behavior of such parameters (variation in time for the specific patient) may be added (as these parameters may fluctuate in time in such a way that creates a patient to patient difference). A disease activity score (DAS), as an example of the disease level, is computed according to e.g. an algorithm as disclosed above.

Two exemplary scenarios shall shows such a DAS value and the fluctuation of the parameters in time can be used to manage the disease activity, i.e. to manage treatment.

In scenario A the patient X has been sleeping badly for a couple of weeks. Consequently, the value of cortisol will likely be high and melatonin likely low. This will also be reflected in the computed DAS. Taking these trends into account the treatment over time, e.g. the administration of the drug over time, is adjusted.

In scenario B the patient X has suffered from a flu for a few days. Consequently, the value of WBC, cytokines and temperature is likely to be higher. This will be reflected in the computed DAS. Taking these trends and DAS variation into account the treatment over time, e.g. the administration of the drug over time, is adjusted.

Thus, the device, system and method in accordance with the present invention are able to determine the best treatment interventions, e.g. the best drug administration routine in terms of timing and peak concentration and its changes over time, in order to maintain the effective treatment, e.g. the effective concentration of drug (that works the best for the specific patient).

The disclosed invention can be used in the disease management of patients during treatment for various inflammatory autoimmune diseases, particularly including rheumatoid arthritis, psoriasis, systemic lupus erythematosus, inflammatory bowel disease, inflammatory myopathies as well as asthma. Further, it may potentially also be used in the treatment of chronic inflammatory diseases, such as chronic fatigue syndrome, inflammatory multiple sclerosis, primary Sjögren's syndrome and Systemic lupus erythematosus.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. Device for managing treatment of an inflammatory autoimmune disease of a person, said device comprising:
an input unit for receiving measurement data acquired over time, wherein the received measurement data are parameters including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration, and
a treatment manager for managing treatment of the inflammatory autoimmune disease of the person by combining the obtained measurement data by an algorithm to obtain a disease activity level, to monitor the disease activity level over time and to determine treatment interventions based on the monitored disease activity level.

2. Device as claimed in claim 1,
wherein said treatment manager is configured to monitor trends over time in at least one, preferably all, of the measurement data.

3. Device as claimed in claim 1,
wherein said treatment manager is configured to combine the obtained measurement data based on a base line value of one or more parameters for the person as well as monitoring and analyzing their trend during treatment time, and to use the combined measurement data, alone or in addition to one or more of the obtained measurement data, for managing treatment.

4. Device as claimed in claim 1, further comprising an interface for issuing treatment information, user information, therapy recommendations and/or decision support.

5. Device as claimed in claim 1,
wherein said treatment manager is further configured to determine one or more of frequency, period, timing and dosage of treatment interventions.

6. Device as claimed in claim 1,
wherein said input unit is further configured to obtain temperature data and/or cytokine data of the person acquired over time and wherein said treatment manager is further configured to additionally use the obtained temperature data and/or cytokine data for managing treatment.

7. Device as claimed in claim 1,
wherein said treatment manager is configured to additionally extract chronobiology information from one or more further parameters related to the chronobiology of the person, the treatment manager being further configured for managing treatment based on said extracted chronobiology information.

8. Device as claimed in claim 1,
wherein said treatment manager is configured to determine for the obtained measurement data a respective deviation of the respective measurement data from a predetermined range, in particular a person-related range, for combining, in particular adding, said deviations and for managing treatment, in particular for scheduling the timing of treatment, based on the combined deviations.

9. Computer-implemented method for managing treatment of an inflammatory autoimmune disease of a person, said method comprising:
receiving measurement data acquired over time including white blood cell count data related to the person's white blood cell count, tumor necrotic factor-α data related to the person's tumor necrotic factor-α, C-reactive protein data related to the person's C-reactive protein, cortisol level data related to the person's cortisol level and melatonin data related to the person's melatonin concentration, and
managing treatment of the inflammatory autoimmune disease of the person by combining the obtained measurement data by an algorithm to obtain a disease activity level, to monitor the disease activity level over time and to determine treatment interventions based on the monitored disease activity level.

10. Method as claimed in claim 9,
wherein the steps of said method are performed on a stationary or mobile apparatus, in particular a smart phone, a mobile phone, a laptop, a computer, a tablet, a watch or a camera.

11. Method as claimed in claim 9,
wherein the steps of said method are implemented in software or an application program.

12. System for managing treatment of an inflammatory autoimmune disease of a person, said system comprising:
a white blood cell counter for counting the white blood cells of the person,
a tumor necrotic factor-α sensor for determining the tumor necrotic factor-α data related to the person's tumor necrotic factor-α,
a cortisol level sensor for determining the cortisol level of the person,
a C-reactive protein sensor for determining C-reactive protein data related to the person's C-reactive protein,
a melatonin sensor for determining melatonin data related to the person's melatonin concentration, and
a device as claimed in claim 1 for managing treatment of an inflammatory autoimmune disease of a person based on the obtained measurement data acquired over time by the white blood cell counter and said sensors.

13. System as claimed in claim 12,
further comprising one or more of a video camera, a microphone, a body wearable sensor and a stationary sensor for obtaining person activity related to one or more activities of the person and/or physiological data related to one or more physiological parameters of the person, wherein said device is configured to additionally use the obtained person activity and/or physiological data for managing treatment.

14. A computer program product comprising a computer readable medium having computer readable code embodied therein, the computer readable code being configured such that, on execution by a suitable computer or processing unit, the computer or processing unit is caused to perform the method of claim 9.

15. Device as claimed in claim 1, wherein the algorithm is DA(t)=F{Σ((a*WBC+g*Cytokines)+(b*HG)+(c*T)+(d*ΔP)+(e*ΔC)+(f*ΔM))}, wherein DA(t) is the disease activity as a function of time in a 24 hour cycle, WBC is the White Blood Cell count, the Cytokines including tumor necrotic factor-α, HG is hemoglobin, T is the temperature, ΔP are the changes in prolactin, ΔC is the change in cortisol concentration, ΔM is the change in melatonin concentration, a to g are coefficients representing the weight of each of the parameters, wherein DA(t) the disease activity level.
